(19) United States
(12) Patent Application Publication
(10) Pub. No.: US 2020/0401210 A1
(43) Pub. Date: Dec. 24, 2020

(54) METHOD AND SYSTEM FOR MODULATING THE SYMPATHETIC AND PARASYMPETHETIC RESPONSES OF A PATIENT

(71) Applicant: FUTURE WORLD HOLDINGS LLC, Burlingame, CA (US)

(72) Inventors: Raghu Bathina, Los Altos Hills, CA (US); Sridhar Prathikanti, Burlingame, CA (US)

(21) Appl. No.: 16/450,982

(22) Filed: Jun. 24, 2019

(51) Int. Cl. G06F 3/01; G06T 19/00; G16H 50/30

(52) U.S. Cl. CPC G06F 3/011; G06F 3/017; G16H 50/30; G06T 19/006; G06T 19/003

(57) ABSTRACT

A method and system for modulating a patient's responses to a medical intervention is provided. The method comprises provisioning a system with immersive content comprising visual content and associating at least one stimulation with the visual content, wherein the stimulation is selected from the group consisting of auditory stimulations, olfactory stimulations, gustatory stimulations, neurological stimulations, environmental stimulations, and tactile stimulations; allowing for the selection of an immersive experience from the immersive content; and responsive to said selection, rendering the immersive experience to the user by providing visual content to the user via a display device, and performing at least one stimulation associated with the visual content.

### FIELD

Embodiments of the present invention relate to a system and method for modulating a patient's sympathetic and parasympathetic response for a medical intervention.

### BACKGROUND

Sympathetic and parasympathetic refers to the biological nervous system of the body whether under routine human regulation or in response to an external stimulus. etc.

Modulating the sympathetic and parasympathetic responses of a patient during a medical procedure has beneficial effects including reducing anxiety in the patient, promoting relaxation, increasing the patients tolerance to pain thereby reducing the amount of anesthetics needed during the medical procedure, etc.

### SUMMARY

Embodiments of the invention disclose a system and method for modulating the sympathetic and parasympathetic responses a patient, for example before, during, and after any medical procedure.

According to one embodiment of the present invention, a system which serves as a neurosensory input device for the human body to modulate sympathetic and parasympathetic responses of the patient using Virtual Reality and Augmented Reality, during insertion or readjustment of native or non-native objects in the human body.

The system may consist of VR and or AR system of a near eye display to project a synthetic 3D scene, into both eyes of a user, to generate a virtual realty environment; and computer-generated images as well as mediated reality referred above. The system may include electric scent devices emitting aromatic scents as well as physical objects that simulate gustatory or taste. The system include haptic sleeves, body wear and gloves that stimulate skin/integumentary reactions all over the body.

In one embodiment, the system include a built-in audio system that may give audio instructions as well as simulating auditory noises such as music, construction, any noises in an urban or rural environment as well as sounds created naturally such as glaciers calving, wind, moving water or any sounds experienced in nature. The system is also unique in allowing proprioception to be gauged to allow the patient to experience various limbs and their location relative to a static starting point. The system's gesture-posture capturing device configured to derive gestures of at least one body part of said user for example turning the head to indicate a direction to navigate in the artificial environment; and a computer processor configured to translate the derived gestures of said body part into a movement or action of said user in said synthetic 3D scene and modify the viewpoint of the user of the virtual reality environment, based on the translated movement or action. The device may be monitored by sentient and non-sentient equipment and integrate the five senses as well as proprioception for one uniform experience whose entire purpose is to decrease the sympathetic and parasympathetic system of the patient.

According to another embodiment of the present invention: a method for navigating in a virtual reality scene, using postures and gestures is provided herein. The method may include the following steps: projecting a synthetic 3D scene, into both eyes of a user, via a near eye display, so as to provide a virtual reality view to the user; identifying at least one gesture or posture carried out by at least one body part of said user. Another sense organ is selected by the patient for the optimal patient engagement and relaxation. Then remaining senses will be mathematically weighted. In our example: An olfactory selection is neither coincidental nor random and the method for such delivery in chronological sequences and employed to engage and invoke deep subcortical memories in a combination of patient selection of preferences combined with co-variate use of the other senses. This grouping is mathematically derived with olfactory senses lead to the strongest recollection of forgotten memories which will then direct the machine algorithm to generate more reinforcing sensory stimulation. For example, if a patient selects one of their most positive memories of being in a rose garden in the fall, the machine algorithm would not select ocean water scenting with accompanying seagulls calling nor a sea swell with strong wind gusts but an earth smell with gentle mist or rain with floral aromatics with warm sun and a very gentle breeze. Olfactory sense has been shown to have the strongest neocortical memory stimulation. This algorithm in this particular case is using olfactory senses first to engage the patient the deepest and then the other following senses will engage the patient further. The deeper the engagement the better the patient will be able to modulate their sympathetic and parasympathetic responses as the medical team continue with their external guidance on or above the skin or corporal internal guidance insertion of a foreign probe/instrumentation/catheter during a medical procedure.

Other aspects of the invention, will be apparent from the written description below.

### BRIEF DESCRIPTION OF THE DRAWINGS

FIG. 1 is a schematic drawing illustrating an implementation of a Patient Response Modulation System in accordance with one embodiment of the invention.

FIG. 2 is a schematic drawing illustrating a representative mapping of secondary stimulation settings to a visual object of an immersive experience, in accordance with one embodiment of the invention.

FIG. 3 shows a flow chart of operations performed to deliver an immersive experience, in accordance with one embodiment of the invention.

FIG. 4 is a block diagram illustrating exemplary components of the Patient Response Modulation System 100, in accordance with one embodiment of the invention.

### DETAILED DESCRIPTION

In the following description, for purposes of explanation, numerous specific details are set forth in order to provide a thorough understanding of the invention.

Reference in this specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not others.

Moreover, although the following description contains many specifics for the purposes of illustration, anyone skilled in the art will appreciate that many variations and/or alterations to said details are within the scope of the present invention. Similarly, although many of the features of the present invention are described in terms of each other, or in conjunction with each other, one skilled in the art will appreciate that many of these features can be provided independently of other features. Accordingly, this description of the invention is set forth without any loss of generality to, and without imposing limitations upon, the invention.

As will be appreciated by one skilled in the art, the aspects of the present invention may be embodied as a system, method or computer program product. Accordingly, aspects of the present invention may take the form of an entirely hardware embodiment, an entirely software embodiment (including firmware, resident software, micro-code, etc.), or an embodiment combining software and hardware aspects that may all generally be referred to herein as a “circuit,” “module,” or “system.” Furthermore, aspects of the present invention may take the form of a computer program product embodied in one or more computer readable medium(s) having computer readable program code embodied thereon.

Embodiments of the present invention disclose a method and system for modulating the sympathetic and parasympathetic responses of a patient. Advantageously, said responses may be modulated before, during, and after any medical procedure.

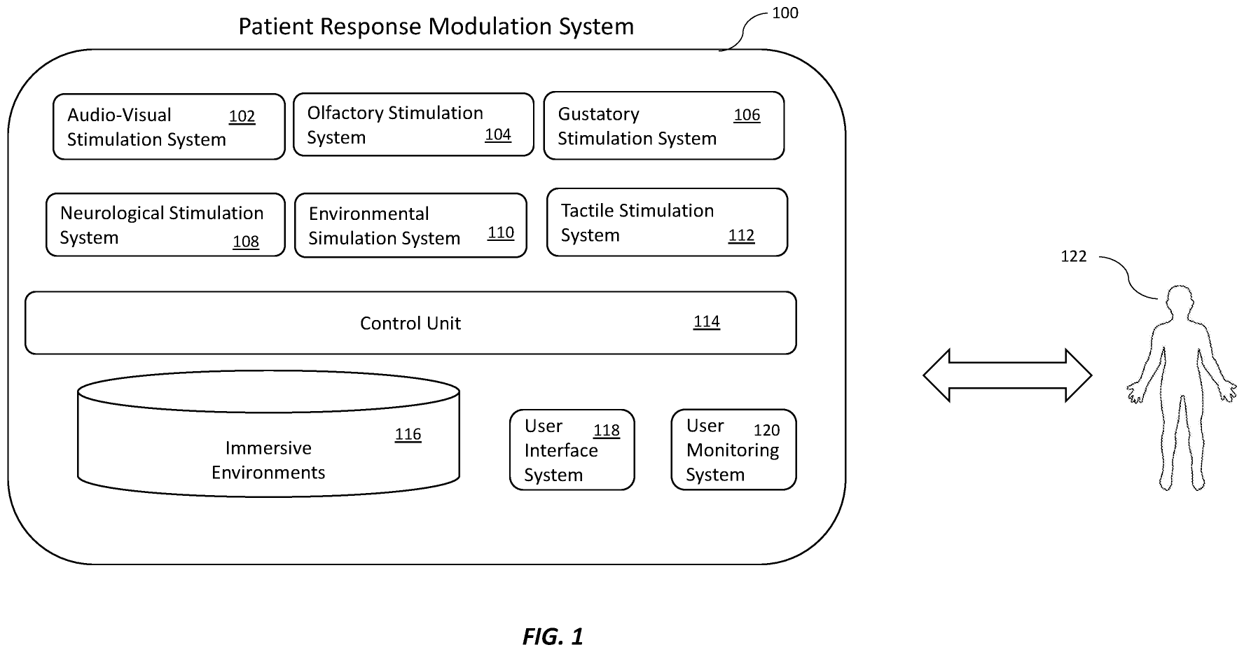

An implementation 100 of the inventive patient response modulation system is shown in FIG. 1 of the drawings. Components of the patient response modulation system 100 comprise an audio-visual stimulation system 102, and olfactory stimulation system 104, a gustatory stimulation system 106, a neurological stimulation system including proprioception 108, an environmental simulation system 110, or tactile stimulation system 112. The aforesaid systems may be used to provide an immersive experience to a user 122, as will be described.

The patient response modulation system 100 further comprises a control unit 114 to control the various components of the system. Database 116 is provisioned with a plurality of immersive experiences which are multisensory in nature and are designed to moderate the sympathetic and parasympathetic responses of the user 122. A user interface system 118 allows the users of the system to interact with the system, consisting 120 comprises a system of sensors to monitor physiological parameters of the user 122.

The audiovisual stimulation system 102 provides the user 122 an audiovisual experience. Advantageously, the audiovisual experience may include content designed to have a calming effect on the user 122. For example, the audiovisual experience may comprise a walk on a beach at sunset, a walk through a forest, or a campfire scene on the beach. In some cases, the audio experience may describe the procedure being performed on the user 122.

In one embodiment, the audiovisual stimulation system 102 may comprise one or multiple devices, including but not limited to a Virtual Reality (VR) headset, Artificial/Augmented Reality (AR), Mixed Reality (MR), hybrid Reality (HR), television(s), monitor(s), projector(s) with projection surface(s), holographic display(s), heads-up display(s), or any other type of visual display.

In one embodiment, the visual experiences comprise static, dynamic, or interactive content. Static content may be content which is simply displayed for the user. Dynamic content may be content that changes over time, but is not influenced by the user's action(s). Interactive content is content that may change in response to the user's action(s). Each visual experience may comprise auditory inputs to be perceived by the user 122. These may be related to the simulation being undertaken, or not; in which case they may be related to objectives set forth by an operator. The auditory inputs may be delivered through audio devices, for example, speakers, headphones, and any other device that may be used to generate auditory stimulation in the user.

In some embodiments, a visual display, and speakers may be integrated into a virtual reality device, for example the virtual reality device sold under the tradename Oculus Go.

The olfactory stimulation system 104 produces an olfactory input for the user 122 in one embodiment, the olfactory stimulation system 104 may be configured to store a plurality of odor molecules in one or more reservoir(s). Dispensing elements in the form of emitters, or other elements may then be provided to release the odor molecules. In one embodiment, the odor molecules may be released in conjunction with the simulation the user is undergoing, thereby to improve the immersive nature of the simulation that the user is undergoing. In one embodiment, the release of the odor molecules may be coordinated with visual elements of the immersive experience. For example, the immersive experience may comprise a walk through a forest, in which case the odor molecules corresponding to the scents associated with selected objects encountered during the walk may be synchronously released by the olfactory stimulation system 104 as the selected objects are encountered by the user 122. Thus, the odor molecules may be used to provide coordinated inputs relating to a particular scene, in order to improve memory, reduce anxiety, reduce depression, and/or improve cognitive function in the user.

The gustatory stimulation system 106 may be operable to simulate tastes in the user 122. The gustatory stimulations may be achieved, for example, through the use of electrical stimulation by one or more electrodes and temperature variations in the tongue, both in order to achieve taste simulation. Advantageously, it may be used to produce a calming effect on the user 122 while the user is undergoing a medical procedure.

The neurological stimulation system 108 provides neurological stimuli to the user 122, for example to reduce anxiety during a medical procedure. The neural stimulation may be provided by the use of electrodes and may include techniques such as Deep Brain Stimulation (DBS), Transcranial Magnetic Stimulation (TMS), and Transcranial Electric Stimulation (TES). As well as incorporating stimuli based on proprioception to orient the user in the virtual world with their neurological feedback.

The environmental stimulation system 110 may be configured to simulate desired environmental conditions including atmospheric conditions. These conditions may be related to the simulation being undertaken, or not; in which case they may be related to objectives set forth by the operator. For controlling the environmental conditions during an immersive experience, the environmental stimulation system 110 may include components such as fans, heaters, air conditioners, humidifiers, dehumidifiers, radiators, mist generators, and spotlights.

The tactile stimulation system 112 reproduces the physical sensations a user perceives in a given simulation environment. These may be related to the simulation being undertaken, or not; in which case they may be related to objectives set forth by the operator. The stimulated sensations, for example, include: pressure, force, vibration, hardness, texture, and temperature of surfaces. The tactile stimulation system 112 may include, wearable actuators, such as haptic gloves, haptic bodysuits, etc. other components of the tactile stimulation system 112 may include electrodes, heaters, chillers, inflatable bladders, servos, ultrasonic actuators, acoustic actuator, Eccentric Rotating Mass (ERM), Linear Resonant Actuator (LRA), Piezoelectric, Electro-Active Polymer (EAP), Shape Memory Alloy (SMA) and any other device that may be used to provide tactile stimulation to the user.

The control unit 114 controls the various components of the patient response modulation system 100 and may include circuits, switches, software, etc. to perform its tasks.

Simulated immersive experiences stored in the database 116 and include a plurality of experiences designed to reduce anxiety in a patient.

The User Interface System 118 comprises an interface for the user 122 to interact with the system. In one embodiment, such interaction may include providing the user 122 with options for immersive experiences, and facilitating the user's input in selecting an immersive experience from the options. Thus, the user interface, may include a display device, and a user input capture device. In some cases, these devices may be incorporated into a single device via a touch interface. A similar interface may be provided to an operator of the system, to configure options associated with the system.

The User Monitoring System 120 monitors user-specific parameters. The parameters may include Heart rate (HR), invasive or non-invasive blood pressure (IBP or NIBP, respectively), galvanic skin response (GSR), respiration rate (RR), respiratory volume (RV), oxygen saturation (SpO2), oxygen perfusion (perf), oxygen consumption, skin color, skin temperature, skin texture, metabolic rate, pupil dilation, blood glucose level (BGL), blood gases, protein levels, electrocardiogram (ECG), electromyograph (EMG), electroencephalogram (EEG), cutis anserine (goosebumps), cardiac output, digestive system function, etc.

The aforementioned parameters, and others, may be monitored and measured by various sensors and methods.

Advantageously, the patient response modulation system 100 may be used to provide a method for a user to navigate a virtual reality scene, using postures and gestures. The method may include the following steps: projecting a synthetic 3D scene, into both eyes of a user, via a near eye display, so as to provide a virtual reality view to the user; identifying at least one gesture or posture carried out by at least one body part of said user. Another sense organ is selected by the patient for the optimal patient engagement and relaxation. The remaining senses may then be mathematically weighted. In some cases, an immersive experience may be statically or dynamically configured. For dynamic configuration, a combination of the user input, and operator input may be used to control settings associated with the immersive experience. For example, one setting may control the predominance of the olfactory sense in the immersive experience. In such cases, the olfactory sense represents the primary sense by which chronological sequences of the immersive experience are delivered to engage and invoke deep subcortical memories. This is important, as research has shown that the olfactory sense plays a primary or dominant role in invoking the strongest recollection of forgotten memories as it provides the strongest neocortical memory stimulation Said immersive experience may be delivered based on a a combination of patient selected preferences together with co-variate use of the other senses. This combination may be mathematically derived with olfactory senses playing a primary a dominant role and a machine algorithm may be configured to generate more complementary or reinforcing sensory stimulation. For example, if a patient selects one of their most positive memories of being in a rose garden in the fall, the machine algorithm would not select ocean water scenting with accompanying seagulls calling nor a sea swell with strong wind gusts but an earth smell with gentle mist or rain with floral aromatics with warm sun and a very gentle breeze. Thus, the machine algorithm in this particular case is configured to use olfactory senses first to engage the patient at the deepest level and then the other following senses will engage the patient further. The deeper the engagement the better the patient will be able to modulate their sympathetic and parasympathetic responses as the medical team continue with their external guidance on or above the skin or corporal internal guidance insertion of a foreign probe/instrumentation/catheter during a medical procedure.

One particular technique to reduce anxiety in a patient involves the use of audio guidance during a medical procedure. Typically, medical procedures are performed, with a patient in a lying position, with medical staff positioned around the patient, each performing a defined task relating to the medical procedure. In all of this, the patient's anxiety tends to increase as they find themselves in an unfamiliar environment, with people performing various tasks, but with little or no knowledge of what is actually happening. To reduce anxiety in such cases, the audio guidance may include details of the actual medical procedure as it is being performed so that the patient understands what is happening. This has a tendency to reduce stress levels in the patient. Thus, in some embodiments, the immersive experiences are designed to include audio input that provides information on the medical procedure being performed in a synchronous manner.

Embodiments of the present invention comprise techniques to generate simulated immersive experiences. As noted, each immersive experience may comprise visual content. This content may comprise video, and/or computer-generated imagery. In one embodiment, the visual content is regarded as the primary component, whereas the other components of the immersive experience such as the components for audio stimulation, gustatory stimulation, olfactory stimulation, neurological stimulation, tactile stimulation, and environmental stimulation are regarded as secondary components. For a high fidelity simulated experience, in one embodiment, a method is provided for coordinating the delivery of the primary component and the secondary components of an immersive experience. In accordance with said method, selected objects of visual content associated with an immersive experience are indexed in a time sequence representing an order for the presentation of said objects to the user. For example, in the case of a guided forest walk, the selected objects of the visual content may include a stream, a pine tree, a stream, and sage brush. For each of these selected objects, a mapping operation is performed to map the secondary components associated with said selected objects. For example, the stream may have a particular audio stimulation, and olfactory stimulation associated with it. These elements will as a result of the mapping operation be mapped to the stream. Thus, when the immersive experience is rendered, the mapping will be used to retrieve and invoke the secondary components thereby to provide a truly immersive experience.

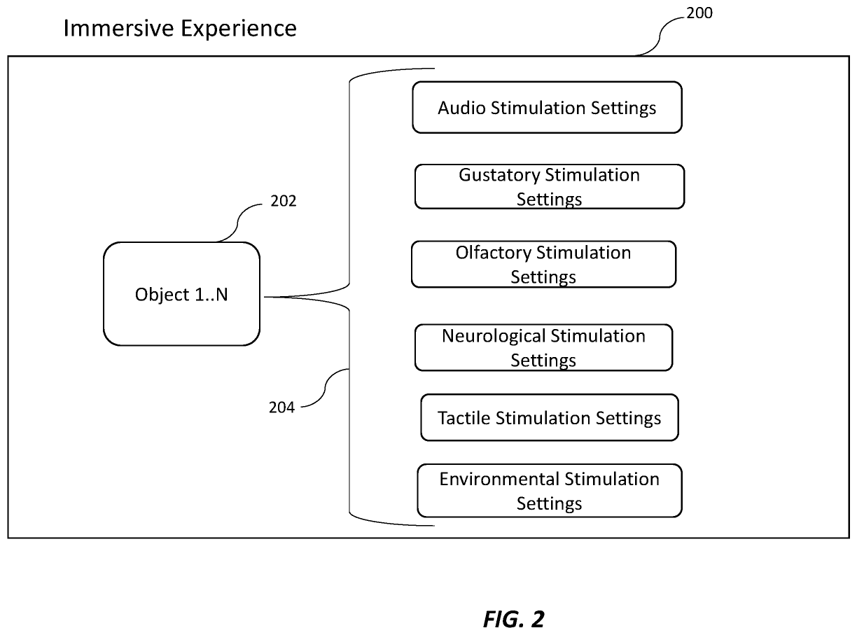

Referring now to FIG. 2 of the drawings, reference 200 indicates an immersive experience comprising a plurality of objects 1 to N indicated by reference 202. In accordance with the techniques outlined above, for each object 1 . . . N, there is provided a mapping 204 comprising the audio stimulation settings, gustatory is to settings, olfactory system relations settings, neurological stimulation settings, tactile stimulation settings, and environmental stimulation settings for the object.

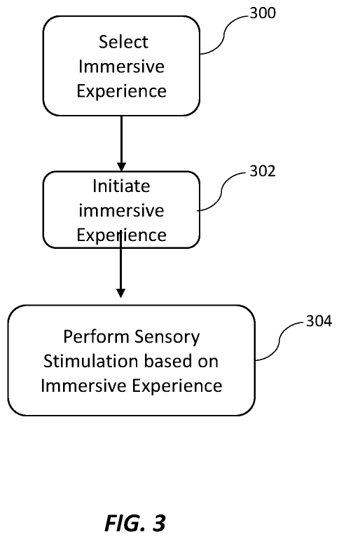

FIG. 3 of the drawings, shows a flow chart of operations performed in order to deliver an immersive experience to a user. To begin, and immersive experience is selected at block 300. This step may be performed by the patient/user using the above-described user interface, or by an operator (typically this will be medical personnel). For example, in one embodiment, the system may be configured to provide a menu of immersive experiences, and the user may be prompted to input a selection from the menu.

At block 302, once the immersive experience has been selected, the system responds by initiating the immersive experience which typically will involve the commencement of delivery of the content associated with the selected immersive experience. Control then passes to the block 304, wherein sensory stimulation based on the selected immersive experience is performed. In one embodiment, the step may include accessing the mapping associated with visual objects in the immersive experience, and then providing the stimulations defined in the mapping.

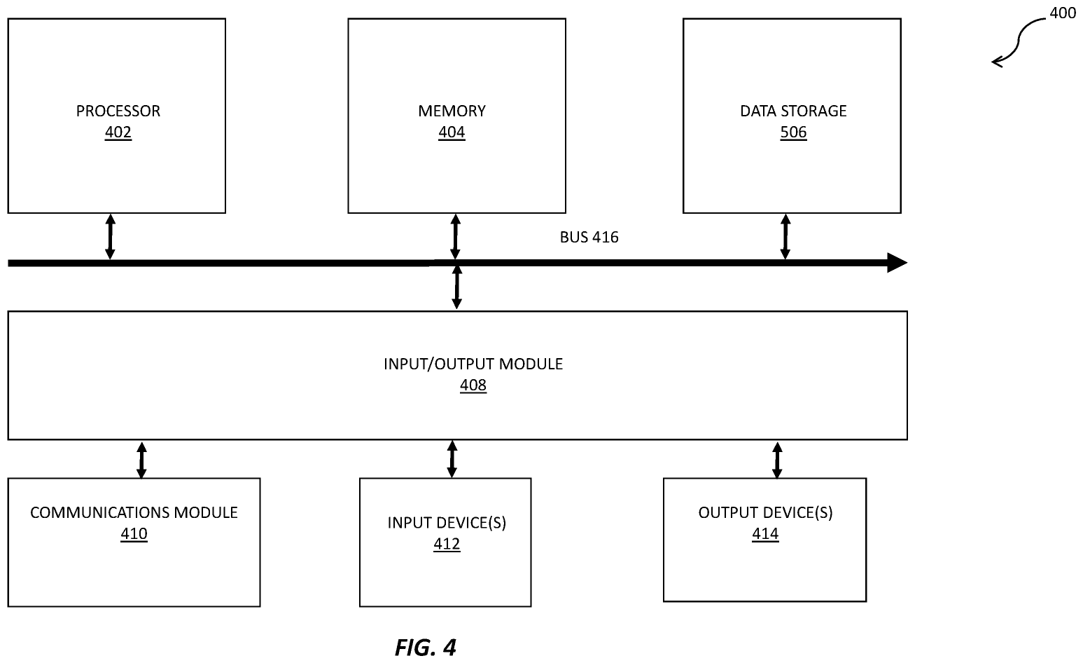

FIG. 4 is a block diagram illustrating exemplary components of the Patient Response Modulation System 100 in the form of a system 400, in accordance with one embodiment of the invention. In certain aspects, the system 400 may be implemented using hardware or a combination of software and hardware, either in a dedicated server or integrated into another entity or distributed across multiple entities.

The system 400 (e.g., client or server) includes a bus 408 or other communication mechanism for communicating information, and a processor 402 coupled with bus 416 for processing information. According to one aspect, the system 400 is implemented as one or more special-purpose computing devices. The special-purpose computing device may be hard-wired to perform the disclosed techniques, or may include digital electronic devices such as one or more application-specific integrated circuits (ASICs) or field programmable gate arrays (FPGAs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, or FPGAs with custom programming to accomplish the techniques. The special-purpose computing devices may be desktop systems, portable systems, handheld devices, networking devices or any other device that incorporates hard-wired and/or program logic to implement the techniques. By way of example, the system 400 may be implemented with one or more processors 402. Processor 402 may be a general-purpose microprocessor, a microcontroller, a Digital Signal Processor (DSP), an ASIC, a FPGA, a Programmable Logic Device (PLD), a controller, a state machine, gated logic, discrete hardware components, or any other suitable entity that can perform calculations or other manipulations of information.

The system 400 can include, in addition to hardware, code that creates an execution environment for the computer program in question, e.g., code that constitutes processor firmware, a protocol stack, a database management system, an operating system, or a combination of one or more of them stored in an included memory, such as a Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, or any other suitable storage device, coupled to bus 416 for storing information and instructions to be executed by processor 402. The processor 402 and the memory 404 can be supplemented by, or incorporated in, special purpose logic circuitry. Expansion memory may also be provided and connected to system 600 through input/output module 408, which may include, for example, a SIMM (Single in Line Memory Module) card interface. Such expansion memory may provide extra storage space for system 400 or may also store applications or other information for system 400. Specifically, expansion memory may include instructions to carry out or supplement the processes described above and may include secure information also. Thus, for example, expansion memory may be provided as a security module for system 400 and may be programmed with instructions that permit secure use of system 400. In addition, secure applications may be provided via the SIMM cards, along with additional information, such as placing identifying information on the SIMM card in a non-hackable manner.

The instructions may be stored in the memory 404 and implemented in one or more computer program products, i.e., one or more modules of computer program instructions encoded on a computer readable medium for execution by, or to control the operation of, the system 400, and according to any method well known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multiparadigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, embeddable languages, and xml-based languages. Memory 404 may also be used for storing temporary variable or other intermediate information during execution of instructions to be executed by processor 402.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors executing one or more computer programs to perform functions by operating on input data and generating output.

System 400 further includes a data storage device 406 such as a magnetic disk or optical disk, coupled to bus 416 for storing information and instructions. System 400 may be coupled via input/output module 408 to various devices mentioned above, such as haptic devices, sensors, electrodes, monitors, etc. In addition, input/output module 408 may be provided in communication with processor 402, so as to enable near area communication of system 400 with other devices. The input/output module 408 may provide, for example, for wired communication in some implementations, or for wireless communication in other implementations, and multiple interfaces may also be used. The input/output module 408 is configured to connect to a communications module 410. Example communications modules 410 include networking interface cards, such as Ethernet cards and modems.

The components of the system can be interconnected by any form or medium of digital data communication, e.g., a communication network. The communication network can include, for example, any one or more of a PAN, a LAN, a CAN, a MAN, a WAN, a BBN, the Internet, and the like. Further, the communication network can include, but is not limited to, for example, any one or more of the following network topologies, including a bus network, a star network, a ring network, a mesh network, a star-bus network, tree or hierarchical network, or the like.

For example, in certain aspects, communications module 410 can provide a two-way data communication coupling to a network link that is connected to a local network. Wireless links and wireless communication may also be implemented. Wireless communication may be provided under various modes or protocols, such as GSM (Global System for Mobile Communications), Short Message Service (SMS), Enhanced Messaging Service (EMS), or Multimedia Messaging Service (MMS) messaging, CDMA (Code Division Multiple Access), Time division multiple access (TDMA), Personal Digital Cellular (PDC), Wideband CDMA, General Packet Radio Service (GPRS), or LTE (Long-Term Evolution), among others. Such communication may occur, for example, through a radio-frequency transceiver. In addition, short-range communication may occur, such as using a BLUETOOTH, WI-FI, or other such transceiver.

In any such implementation, communications module 410 sends and receives electrical, electromagnetic or optical signals that carry digital data streams representing various types of information. The network link typically provides data communication through one or more networks to other data devices. For example, the network link of the communications module 410 may provide a connection through local network to a host computer or to data equipment operated by an Internet Service Provider (ISP). The ISP in turn provides data communication services through the world-wide packet data communication network now commonly referred to as the Internet. The local network and Internet both use electrical, electromagnetic or optical signals that carry digital data streams. The signals through the various networks and the signals on the network link and through communications module 610, which carry the digital data to and from system 600, are example forms of transmission media.

System 440 can send messages and receive data, including program code, through the network(s), the network link and communications module 410. In the Internet example, a server might transmit a requested code for an application program through Internet, the ISP, the local network and communications module 410. The received code may be executed by processor 402 as it is received, and/or stored in data storage 606 for later execution.

In certain aspects, the input/output module 408 is configured to connect to a plurality of devices, such as an input device 412 and/or an output device 414. Example input devices 412 include a stylus, a finger, a keyboard and a pointing device, e.g., a mouse or a trackball, by which a user can provide input to the system 400. Other kinds of input devices 412 can be used to provide for interaction with a user as well, such as a tactile input device, visual input device, audio input device, or brain-computer interface device. For example, feedback provided to the user can be any form of sensory feedback, e.g., visual feedback, auditory feedback, or tactile feedback; and input from the user can be received in any form, including acoustic, speech, tactile, or brain wave input. Example output devices 414 include display devices, such as a LED (light emitting diode), CRT (cathode ray tube), LCD (liquid crystal display) screen, a TFT LCD (Thin-Film-Transistor Liquid Crystal Display) or an OLED (Organic Light Emitting Diode) display, for displaying information to the user. The output device 414 may comprise appropriate circuitry for driving the output device 414 to present graphical and other information to a user.

According to one aspect of the present disclosure, the techniques disclosed herein may be implemented in response to processor 402 executing one or more sequences of one or more instructions contained in memory 404. Such instructions may be read into memory 404 from another machine-readable medium, such as data storage device 406. Execution of the sequences of instructions contained in main memory 404 causes processor 402 to perform the process steps described herein. One or more processors in a multi-processing arrangement may also be employed to execute the sequences of instructions contained in memory 404. In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component, e.g., a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components.

The term “machine-readable storage medium” or “computer-readable medium” as used herein refers to any medium or media that participates in providing instructions or data to processor 402 for execution. The term “storage medium” as used herein refers to any non-transitory media that store data and/or instructions that cause a machine to operate in a specific fashion. Such a medium may take many forms, including, but not limited to, non-volatile media, volatile media, and transmission media. Non-volatile media include, for example, optical disks, magnetic disks, or flash memory, such as data storage device 406. Volatile media include dynamic memory, such as memory 404. Transmission media include coaxial cables, copper wire, and fiber optics, including the wires that comprise bus 416. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter affecting a machine-readable propagated signal, or a combination of one or more of them.

As used in this specification of this application, the terms “computer-readable storage medium” and “computer-readable media” are entirely restricted to tangible, physical objects that store information in a form that is readable by a computer. These terms exclude any wireless signals, wired download signals, and any other ephemeral signals. Storage media is distinct from but may be used in conjunction with transmission media. Transmission media participates in transferring information between storage media. For example, transmission media includes coaxial cables, copper wire and fiber optics, including the wires that comprise bus 416. Transmission media can also take the form of acoustic or light waves, such as those generated during radio-wave and infra-red data communications. Furthermore, as used in this specification of this application, the terms “computer”, “server”, “processor”, and “memory” all refer to electronic or other technological devices. These terms exclude people or groups of people. For the purposes of the specification, the terms display or displaying means displaying on an electronic device.

To illustrate the interchangeability of hardware and software, items such as the various illustrative blocks, modules, components, methods, operations, instructions, and algorithms have been described generally in terms of their functionality. Whether such functionality is implemented as hardware, software or a combination of hardware and software depends upon the particular application and design constraints imposed on the overall system. Skilled artisans may implement the described functionality in varying ways for each particular application.

As used herein, the phrase “at least one of” preceding a series of items, with the terms “and” or “or” to separate any of the items, modifies the list as a whole, rather than each member of the list (i.e., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

To the extent that the term “include,” “have,” or the like is used in the description or the claims, such term is intended to be inclusive in a manner similar to the term “comprise” as “comprise” is interpreted when employed as a transitional word in a claim. Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

A reference to an element in the singular is not intended to mean “one and only one” unless specifically stated, but rather “one or more.” The term “some” refers to one or more. Underlined and/or italicized headings and subheadings are used for convenience only, do not limit the subject technology, and are not referred to in connection with the interpretation of the description of the subject technology. Relational terms such as first and second and the like may be used to distinguish one entity or action from another without necessarily requiring or implying any actual such relationship or order between such entities or actions. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description. No claim element is to be construed under the provisions of 35 U.S.C. § 112, sixth paragraph, unless the element is expressly recited using the phrase “means for” or, in the case of a method claim, the element is recited using the phrase “step for.”

While this specification contains many specifics, these should not be construed as limitations on the scope of what may be claimed, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products.

The title, background, brief description of the drawings, abstract, and drawings are hereby incorporated into the disclosure and are provided as illustrative examples of the disclosure, not as restrictive descriptions. It is submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the claimed subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately claimed subject matter.

The claims are not intended to be limited to the aspects described herein but are to be accorded the full scope consistent with the language claims and to encompass all legal equivalents. Notwithstanding, none of the claims are intended to embrace subject matter that fails to satisfy the requirements of the applicable patent law, nor should they be interpreted in such a way.

1. A method for modulating a sympathetic and parasympathetic response in a patient, the method comprising:
provisioning a system with immersive content comprising visual content and associating at least one stimulation with the visual content, wherein the stimulation is selected from the group consisting of auditory stimulations, olfactory stimulations, gustatory stimulations, neurological stimulations, environmental stimulations, and tactile stimulations;
allowing for the selection of an immersive experience from the immersive content; and
responsive to said selection, rendering the immersive experience to the user comprising providing the visual content to the user via a display device, and performing the at least one stimulation associated with the visual content.

2. The method of claim 1, wherein performing the at least one stimulation comprises synchronizing said stimulation with the presentation of the visual content to the user.

3. The method of claim 1, further comprising dynamically configuring settings associated with the selected immersive experience.

4. The method of claim 3, wherein said settings comprise a setting to emphasize olfactory stimulation when rendering the immersive experience over other stimulations associated with the immersive experience.

5. The method of claim 1, wherein auditory stimulation associated with the immersive experience comprises spoken guidance in the form of explanations relating to a medical procedure performed on the user.

6. A system for modulating a sympathetic and parasympathetic response in a patient, the system comprising:
a database for storing immersive content comprising visual content and associating at least one stimulation with the visual content, wherein the stimulation is selected from the group consisting of auditory stimulations, olfactory stimulations, gustatory stimulations, neurological stimulations, environmental stimulations, and tactile stimulations;
a user interface allowing for the selection of an immersive experience from the immersive content; and
a control unit configured to generate instructions for rendering the visual immersive experience to the user comprising providing the visual content to the user via a display device, and performing the at least one stimulation associated with the visual content, responsive to said selection.

7. The system of claim 6, wherein performing the at least one stimulation comprises synchronizing said stimulation with the presentation of the visual content to the user.

8. The system of claim 6, the control unit is adapted to dynamically configure settings associated with the selected immersive experience.

9. The system of claim 8, wherein said settings comprise a setting to emphasize olfactory stimulation when rendering the immersive experience over other stimulations associated with the immersive experience.

10. The system of claim 6, wherein auditory stimulation associated with the immersive experience comprises spoken guidance in the form of explanations relating to a medical procedure performed on the user.
